(19) United States
(12) Patent Application Publication
(10) Pub. No.: US 2025/0197798 A1
(43) Pub. Date: Jun. 19, 2025

(54) METHOD OF CULTIVATING AND EXTRACTING CALLUS OF DAMNACANTHUS MAJOR AND COSMETIC COMPOSITION COMPRISING DAMNACANTHUS MAJOR CALLUS PREPARED BY USING THE SAME

(71) Applicant: UCL CO., LTD., Jeju-si (KR)

(72) Inventors: Eun Bi Jang, Chungcheongbuk-do (KR); Boram Go, Jeju-do (KR); Sung Chun Kim, Jeju-do (KR); Young-Min Ham, Jeju-do (KR); Seon-A Yoon, Jeju-do (KR); Hyejin Hyeon, Jeju-do (KR); Yu-Jung Han, Jeju-do (KR); Jomg-Do Lee, Jeju-do (KR); Ho Bong Hyun, Jeju-do (KR); Yong-Hwan Jung, Jeju-do (KR); Jeong Mi Kim, Jeju-do (KR); Ji Young Moon, Jeju-do (KR); Kee-Yoeup Paek, Chungcheongbuk-do (KR); A-Reum Kwon, Chungcheongbuk-do (KR)

(21) Appl. No.: 18/980,879

(22) Filed: Dec. 13, 2024

(30) Foreign Application Priority Data
Dec. 13, 2023 (KR) 10-2023-0180395

(51) Int. Cl. C12N 5/04

(52) U.S. Cl. CPC C12N 5/04

(57) ABSTRACT

The present disclosure provides a method for culturing the callus of Damnacanthus major, which includes a first culturing step of inoculating a culture medium with the callus of Damnacanthus major and culturing Damnacanthus major callus cells; and a second culturing step of sequentially treating the callus of Damnacanthus major with methyl jasmonate as an elicitor and culturing the same, and a method for extracting active ingredients from the cultured Damnacanthus major callus cells. Through the culturing method and extraction method, the active ingredient digiferruginol-11-O-β-primeveroside can be effectively obtained from the callus of Damnacanthus major.

### CROSS-REFERENCE TO RELATED APPLICATION

This application claims priority of Korean Patent Application No. 10-2023-0180395, filed on Dec. 13, 2023, in the KIPO (Korean Intellectual Property Office), the disclosure of which is incorporated herein entirely by reference.

### BACKGROUND OF THE INVENTION

### Field of the Invention

The present disclosure relates to a technology for extracting a natural product, more specifically, to a technology for culturing and extraction for enhancing active ingredients of a Damnacanthus major callus extract.

### Description of the Related Art

Damnacanthus major is an evergreen shrub of the family Rubiaceae. It is native to warm temperate zones such as Jeju Island in Korea or southern Japan. It is 30 to 60 cm in height, and has oval or egg-shaped opposite leaves. It is used for ornamental purposes, and its root is also used as a cosmetic ingredient having moisturizing effect since it contains an anthraquinone-based compound.

Plant “callus” is a soft cell tissue formed in the wound area of a plant. It is a mass of undifferentiated, unorganized cells and is also called the plant stem cells. In general, the cells that can redifferentiate in the plant body can divide very quickly or remain partially undifferentiated like cells in meristematic tissues.

Although it is known that the callus of Damnacanthus major contains active ingredients such as polyphenols, flavonoids, etc., the Damnacanthus major callus is not being used actively due to the very low extraction efficiency of these active substances.

Various and improved methods for processing and extraction of the Damnacanthus major callus, as well as precise analysis of the active ingredients in the extract, are required for the active use of Damnacanthus major, which is an endemic plant in Jeju, especially the callus of Damnacanthus major.

### SUMMARY OF THE INVENTION

The present disclosure seeks to enhance the use of the active ingredients in the callus of Damnacanthus major, which is an endemic plant in Jeju, and is directed to providing a method for culturing the callus of Damnacanthus major that can significantly improve the extraction efficiency of the active ingredients in the Damnacanthus major callus.

The present disclosure is also directed to providing a method for preparing an effective extract from the cultured Damnacanthus major callus cells.

The present disclosure is also directed to providing a Damnacanthus major callus extract obtained through the culturing and extraction methods described above and having significantly increased contents of active ingredients.

The present disclosure is also directed to providing a cosmetic composition containing the Damnacanthus major callus extract, having a digiferruginol-11-O-β-primeveroside content of at least 1.8 mg/g in the extract.

A method for culturing the callus of Damnacanthus major according to an exemplary embodiment of the present disclosure includes: a first culturing step of inoculating a culture medium with the callus of Damnacanthus major and culturing Damnacanthus major callus cells; and a second culturing step of sequentially treating the callus of Damnacanthus major with methyl jasmonate as an elicitor and culturing the same.

The culture medium provides a culture environment containing a MS (Murashige & Skog) salt, a plant growth regulator, and sucrose.

It is advantageous that the first culturing step is performed at a temperature of 24 to 26° C.

The plant growth regulator may include α-naphthaleneacetic acid or kinetin, and these compounds may be used simultaneously.

Specifically, the pH of the culture medium may be adjusted to 5.7 to 5.8 throughout the first culturing step and the second culturing step.

Specifically, the methyl jasmonate may be supplied to the culture medium so that the concentration of the methyl jasmonate in the culture medium is maintained at 70 to 150 μM.

A method for preparing a Damnacanthus major callus extract according to an exemplary embodiment of the present disclosure includes: a step of separating a Damnacanthus major callus extract by immersing the Damnacanthus major callus obtained by the culturing method described above in an alcohol having a concentration of 60 to 80%; and a step of decompression-concentrating and freeze-drying the separated callus extract.

According to an exemplary embodiment of the present disclosure, the Damnacanthus major callus extract is obtained by the culturing method and extraction method described above, and the content of digiferruginol-11-O-β-primeveroside in the extract is at least 1.8 mg/g.

According to a method for culturing the callus of Damnacanthus major according to the present disclosure, it is possible not only to maximize the growth of Damnacanthus major callus cells, but also to dramatically increase the accumulation of active ingredients such as polyphenols, flavonoids, etc. within the cells. Therefore, it is possible to secure Damnacanthus major callus cells with enhanced active ingredients.

Furthermore, by providing an extraction condition that can maximize the active ingredients of the callus of Damnacanthus major, the present disclosure allows preparation of an extract having significantly increased contents of active ingredients from the grown Damnacanthus major callus cells as compared to the conventional technology.

The extract obtained by the above culturing and extraction methods can be used as a raw material for a cosmetic composition, particularly a cosmetic product for moisturization. It is expected that it will be possible to develop a cosmetic with dramatically improved moisturizing power as compared to the conventional cosmetics derived from natural products by utilizing the extract of the present disclosure.

In commercial aspect, it will be possible to contribute to the development of the plant industry in Jeju Island by increasing the usefulness and value of Damnacanthus major, which is native to Jeju Island.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a flowchart conceptually showing a method for preparing a Damnacanthus major callus extract according to an exemplary embodiment of the present disclosure.

FIG. 2 schematically shows the separation of active ingredients.

FIG. 3 shows a result of HPLC chromatogram analysis of extracts obtained in examples and comparative examples.

FIG. 4 shows a result of investigating cell viability and hyaluronic acid synthesis in Test Example 5.

FIG. 5 shows a result of evaluating the synthesis of hyaluronic acid depending on the concentration of digiferruginol-11-O-β-primeveroside isolated in Test Example 3.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

Hereinafter, a method for preparing a Damnacanthus major callus extract according to an exemplary embodiment of the present disclosure will be described in detail referring to the attached drawings. A method for culturing the callus of Damnacanthus major will also be described. Furthermore, the obtained Damnacanthus major callus extract and the utilization thereof will also be described. The following description is exemplary explanation for specifying and explaining the technical idea of the present disclosure, and the technical idea of the present disclosure is not limited by the following description. The technical idea of the present disclosure can only be interpreted and limited by the scope of the claims described below.

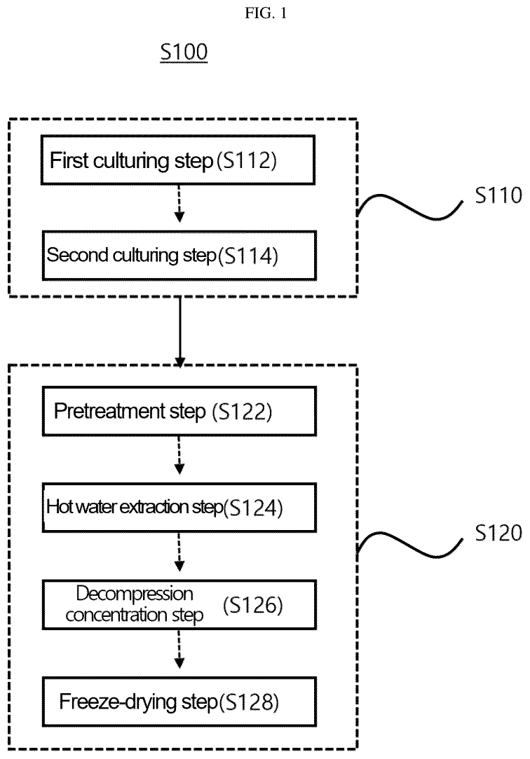

FIG. 1 is a flowchart conceptually showing a method for preparing a Damnacanthus major callus extract according to an exemplary embodiment of the present disclosure.

Referring to FIG. 1, a method for preparing a Damnacanthus major callus extract S100 includes a step of culturing the callus of Damnacanthus major S110 and a step of extracting a Damnacanthus major callus S120. The Damnacanthus major callus extraction step S120 is a step wherein a Damnacanthus major callus extract is substantially prepared.

The Damnacanthus major callus culturing step S110 includes a first culturing step S112 and a second culturing step S114 as a continuous process. The first culturing step S112 is a step of inoculating the callus of Damnacanthus major to a culture medium and growing the same. In this exemplary embodiment, the culture medium contains a MS (Murashige & Skog) salt, a plant growth regulator, and sucrose.

The MS salt is a known culture medium developed in 1962 by American plant physiologists Murashige and Skoog. It is composed of minerals and organic materials and supplies nutrients necessary for plant growth.

As the plant growth regulator, α-naphthaleneacetic acid (NAA) or kinetin can be used. In this exemplary embodiment, the two components may be used simultaneously. NAA is a type of auxin (plant hormone) and is a component which promotes cell division, cell elongation, callus formation, root differentiation, etc. in plants. Kinetin is a compound in the cytokinin group and plays a role in promoting cell division and differentiation. The plant growth regulator can be contained in a culture medium at a content of 0.1 to 1.0%. In this exemplary embodiment, α-naphthaleneacetic acid and kinetin are mixed at a weight ratio of 1:0.3 to 0.5 for use as the plant growth regulator.

The sucrose is a type of sugar. It is a disaccharide composed of glucose and galactose. In this exemplary embodiment, by including the sucrose in the culture medium, cell wall formation is promoted and cell differentiation and growth are assisted in the callus of Damnacanthus major.

After inoculating the callus of Damnacanthus major to the culture medium with the composition described above, the first culturing step S112 is performed.

The first culturing step S112 can be carried out for about 2 to 3 weeks, but the period required for the first culturing step S112 can be set variably in consideration of the purpose of use of the Damnacanthus major callus cells and the characteristics of the Damnacanthus major callus cells.

After the first culturing step S112 is completed, the second culturing step S114 is performed continuously. The second culturing step S114 is a step in which the grown Damnacanthus major callus cells are treated with methyl jasmonate (MJ) and cultured. The methyl jasmonate acts as an elicitor. In the second culturing step S114, the concentration of methyl jasmonate is important. The methyl jasmonate is supplied continuously to the culture medium so that the concentration of the methyl jasmonate in the culture medium is maintained at 70 to 150 μM. However, the essential technical idea of the present disclosure is not limited by the concentration range described above.

The second culturing step S114 lasts approximately 1 week following the first culturing step S112, but this period can also be changed.

The culturing step S110 takes place in an environment strictly controlled at 24 to 26° C., and the pH of the culture medium is specifically controlled at 5.7 to 5.8.

After the culturing step S110 is completed, a Damnacanthus major callus extraction step S120 wherein an effective Damnacanthus major callus extract is prepared from the Damnacanthus major callus cells obtained in the culturing step S110 is performed.

The Damnacanthus major callus extraction step S120 includes a pretreatment step S122, a hot water extraction step S124, a decompression concentration step S126, and a freeze-drying step S128.

First, a pretreatment step S122 wherein the Damnacanthus major callus cells obtained in the culturing step S110 is filtered with a wicker tray to separate the culture medium and moisture is removed sufficiently is performed. The degree of moisture removal (drying) can be determined appropriately in consideration of the usage pattern of the Damnacanthus major callus extract. It is obvious that various known additional pretreatment processes required for subsequent processing can be performed.

In the hot water extraction step S124, the prepared Damnacanthus major callus cells are leached by immersing in purified water. The hot water extraction step S124 can be performed for 3 to 5 hours at a temperature of about 80 to 90° C. After the hot water extraction step S124 is completed, only the filtrate is taken from the leached sample using a suction filter, etc.

The obtained filtrate can be prepared into an extract through a decompression concentration step S126 and a freeze-drying step S128. The decompression concentration step S126 and the freeze-drying step S128 can be performed using various known methods and devices. Specifically, the decompression concentration step S126 may be performed at a temperature of 35 to 45° C.

As a result of NMR analysis, the Damnacanthus major callus extract obtained by the method described above was confirmed to contain digiferruginol-11-O-β-primeveroside. The content of the digiferruginol-11-O-β-primeveroside in the extract was found to be at least 1.8 mg/g. Specifically, the content of digiferruginol-11-O-β-primeveroside in the extract is 1.8 to 2.0 mg/g.

Since the content of digiferruginol-11-O-β-primeveroside in the extract is significantly improved as compared to the conventional technology, the extract can be used very effectively as an active ingredient in moisturizing cosmetic compositions. Meanwhile, it was confirmed that the hyaluronic acid content in the extract was also improved significantly. Details will be explained in the examples and test examples described below.

Since it was confirmed that the Damnacanthus major callus extract obtained through the Damnacanthus major callus culturing step (S110) and the Damnacanthus major callus extraction step (S120) according to an exemplary embodiment of the present disclosure contains highly enhanced active ingredients, it is expected that its utility will be very great.

In the following, specific examples will be used to explain the culturing method and extraction method described above in more detail. Furthermore, the characteristics of the products obtained through the culturing and extraction methods will be examined in detail through analysis of various experiment results.

### EXAMPLES

### Example 1: Culturing of Callus of Damnacanthus major (1)

Two-step culturing was performed in a 5-L bioreactor to maximize growth and accumulation of active ingredients simultaneously. In the first step, 1.5 to 2.5 L of a culture medium containing a MS (Murashige & Skog) salt, a plant growth regulator (α-naphthaleneacetic acid; NAA 3.0 mg/L and kinetin 0.1 mg/L), and sucrose (30 g/L) was used. The pH of the culture medium was adjusted to 5.7 to 5.8. The inoculation dose of the callus was 70 g/L, air supply rate was 0.1 vvm, and culturing temperature was maintained at 25±1° C. The culturing was carried out for approximately 2 weeks. In the two-step culturing for accumulation of active ingredients such as polyphenols, flavonoids, etc., methyl jasmonate (MJ) was treated to Damnacanthus major callus cells, which had been grown for 2 weeks, as an elicitor. Methyl jasmonate was treated for 1 week, from 2 weeks after starting the culturing. It was supplied at a concentration of 100 μM.

### Example 2: Culturing of Callus of Damnacanthus major (2)

The callus of Damnacanthus major was cultured using the same method as in Example 1, except that methyl jasmonate was treated at a concentration 200 μM.

### Comparative Example 1: Culturing of Callus of Damnacanthus major (3)

The callus of Damnacanthus major was cultured using the same method as in Example 1, except that the treatment with methyl jasmonate was omitted.

### [Test Example 1] Analysis of Biomass Indices

The biomass indices of the Damnacanthus major callus cells cultured through the two steps were analyzed. Fresh weight was measured after separating the harvested cells from the culture medium by filtering with a stainless steel wicker tray and sufficiently removing moisture with a clean tissue. After drying the cells at 40° C. for 2 days and measuring weight, the percentage of dry matter was calculated from the percentage of the dry weight to the fresh weight. Also, the growth index and relative growth index of the callus were calculated using Equation 1 and Equation 2.

$$Growth index = [ ( dry weight of finally grown callus ) - ( dry weight of initially inoculated callus ] / ( dry weight of initially inoculated callus ) \qquad [ Equation l ]$$

$$Relative growth index = [ natural logarithm ( dry weight of finally grown callus ) - natural logarithm ( dry weight of initially inoculated callus ) ] / ( culturing period , 3 weeks ) \qquad [ Equation 2 ]$$

The index analysis result for the Damnacanthus major callus cells obtained in Examples 1 and 2 and Comparative Example 1 is shown in Table 1 below.

TABLE 1

| | | | | | Relative |
|---|---|---|---|---|---|
| | Fresh | Dry | Percentage of | Growth | growth |
| Sample | weight (g) | weight (g) | dry matter (%) | index | index |
| Comparative | 530.0 | 37.0 | 7.0 | 9.1 | 0.8 |
| Example 1 | | | | | |
| Example 1 | 334.0 | 28.0 | 8.4 | 6.7 | 0.7 |
| Example 2 | 295.0 | 25.0 | 8.5 | 5.8 | 0.6 |

### Example 3: Extraction of Damnacanthus major Callus (1)

The Damnacanthus major callus cells obtained in Example 1 were dried and crushed, and the crushed product was immersed in distilled water at 90° C. and leached for 4 hours. Only the filtrate was taken from the sample using a suction filter. The filtrate was decompression-concentrated in a 40° C. aqueous solution and then freeze-dried to obtain an extract.

### Example 4: Extraction of Damnacanthus major Callus (2)

A Damnacanthus major callus extract was obtained by the same method as in Example 3, except that the Damnacanthus major callus cells obtained in Example 2 were used.

### Comparative Example 2: Extraction of Damnacanthus major Callus (3)

A Damnacanthus major callus extract was obtained by the same method as in Example 3, except that the treatment with methyl jasmonate was omitted.

### Comparative Example 3: Extraction of Wild Damnacanthus major Leaf

Damnacanthus major leaves collected from the wild were dried and crushed. The crushed leaves were immersed in distilled water at 90° C. and leached for 4 hours. Only the filtrate was taken from the sample using a suction filter. The filtrate was decompression-concentrated in a 40° C. aqueous solution and then freeze-dried to obtain an extract.

### [Test Example 2] Analysis of Extraction Yield

The result of analyzing yield after the extraction according to the methods described in Examples 3 and 4, and Comparative Examples 2 and 3 is shown in Table 2.

TABLE 2

| | Extraction | Extract | |
|---|---|---|---|
| Sample | amount (g) | amount (g) | Yield (%) |
| Comparative Example 2 | 10.0192 | 3.4386 | 34.3 |
| Example 3 | 5.0068 | 1.2306 | 24.6 |
| Example 4 | 5.0102 | 1.4258 | 28.5 |
| Comparative Example 3 | 10.0669 | 1.6901 | 16.8 |

### [Test Example 3] Isolation of Active Ingredients

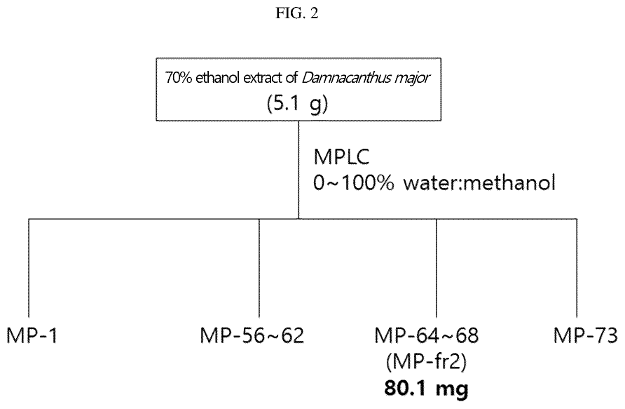

The extract of the Damnacanthus major callus cultured by treating with 100 UM of methyl jasmonate (Example 3) was subdivided according to MPLC polarity. 5.1 g of the extract was dissolved in 10 mL of methanol, filtered using a 0.45-μm PTFE syringe filter, and then injected into a C18 column. Water (solvent A) and methanol (solvent B) were used as a mobile phase, and the flow rate was 15 mL/min. Elution was conducted for 70 minutes with a gradient mode from 10 to 90% of the solvent B. A total of 73 fractions (Fr. MP 1-73) were obtained by eluting with the solvent B (100%) for 10. Among the MPLC fractions, Fr. MP 64-68 (80.1 mg) were identified as single compounds. FIG. 2 schematically shows the separation of active ingredients.

Nuclear magnetic resonance (NMR) for structural analysis was performed using JNM-ECX 400 (FT-NMR system, JEOL, Japan), and DMSO-d6 (CIL) was used as a solvent for the NMR measurement. In order to identify the three-dimensional structure of the compounds isolated from the Damnacanthus major callus, the data on hydrogen and carbon in the molecules were obtained from 1H and 13C-NMR spectra and then compared with the literature. As a result, the compound was identified as digiferruginol-11-O-β-primeveroside. The NMR result of the compound obtained from the experiment is as follows, and the structure of the compound confirmed by the 1H and 13C NMR analysis of the isolated compound is shown in Chemical Formula 1.

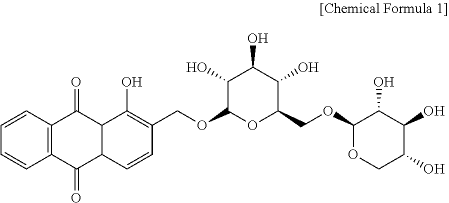

### [NMR Result]

Compound of Chemical Formula 1: yellow, powder; 1H NMR (400 MHZ, DMSO-d6): 8.24 (1H, m, H-8), 8.20 (1H, m, H-5), 8.04 (1H, d, J=7.79 Hz, H-3), 7.95 (2H, m, H-6, 7), 7.74 (1H, d, J=7.79 Hz, H-4), 4.94 (1H, d, J=14.6 Hz, H-11), 4.77 (1H, d, J=14.6 Hz, H-11), 4.33 (1H, d, J=7.79 Hz, H-1′), 4.24 (1H, d, J=7.79 Hz, H-1″), 3.06-3.95 (4H, m, H-2′-H6′), 3.06-3.95 (4H, m, H-2″-H6″); 13C NMR (100 MHZ, DMSO-d6): 188.58 (C-9), 181.77 (C-10), 158.71 (C-1), 135.21 (C-3), 135.07 (C-7), 134.65 (C-6), 133.89 (C-2), 133.17 (C-8a), 132.77 (C-10a), 131.90 (C-4a), 126.86 (C-5), 126.60 (C-8), 118.61 (C-4), 115.20 (C-9a), 103.94 (C-1′), 102.55 (C-1′), 76.53 (C-3″), 76.50 (C-3′), 75.96 (C-5′), 73.43 (C-2′), 73.33 (C-2″), 69.86 (C-4′), 69.55 (C-4″), 68.26 (C-6′), 65.66 (C-5″), 64.07 (C-11).

### [Test Example 4] Analysis of Content of Digiferruginol-11-O-β-Primeveroside

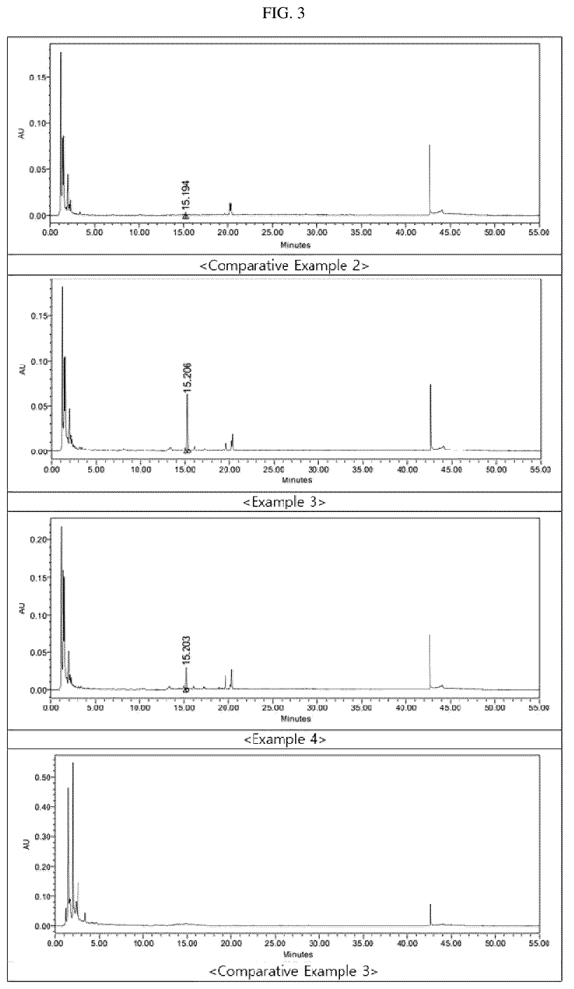

Digiferruginol-11-O-β-primeveroside (70% purity), isolated from the samples of Examples 3 and 4 and Comparative Examples 2 and 3, was dissolved in DMSO to 10,000 ppm. Afterwards, it was diluted to 100, 50, 25, 12.5, 6.25, and 3.12 ppm using 50% methanol to create a calibration curve, and then filtered using a 0.5-μm syringe filter. The extract of Test Example 2 was precisely weighed, prepared to 10,000 ppm by adding H2O, and sonicated for 5 minutes. After filtering with a 0.5-μm syringe filter, the extract was used for analysis. The content of rubiadin was analyzed using the high-performance liquid chromatography (HPLC) system e2695 (Waters Co., Milford, MA, USA) equipped with the Waters photodiode array (PDA) detector 2998 (Waters Co., Milford, MA, USA), and the Sunfire C18 column (4.6×150 mm, 5 μm; Waters Co.) was used for separation of components. The column temperature was maintained at 40° C., and the sample injection volume was 10 μL. Solvent A (0.1% phosphoric acid in water) and solvent B (acetonitrile) were used as a mobile phase, and the flow rate was constant at 1 mL/min. The gradient condition of the mobile phase was as follows: 0 min, 80% A/20% B; 10 min, 80% A/20% B; 30 min, 0% A/100% B; 40 min, 0% A/100% B; 41 min, 80% A/20% B; 50 min, 80% A/20% B. The UV spectrum range used for observation was 200 to 600 nm, and the measurement was made at a wavelength of 260 nm. FIG. 3 shows a result of HPLC chromatogram analysis of extracts obtained in examples and comparative examples.

The result of the analysis of the digiferruginol-11-O-β-primeveroside content is shown in Table 3.

TABLE 3

| | Content of digiferruginol-11-O-β- |
|---|---|
| Sample | primeveroside (mg/g) |
| Damnacanthus major callus MJ_0 | 0.04 |
| Damnacanthus major callus MJ_100 | 1.84 |
| Damnacanthus major callus MJ_200 | 0.79 |
| Wild Damnacanthus major leaf | Not detected |

### [Test Example 5] Analysis of Hyaluronic Acid Synthesis

To measure cell viability, HaCaT cells were dispensed in a 24-well plate at 5×105 cells/well and, after incubation for 24 hours, incubated with the sample at different concentrations for 24 hours. After treating the cultured cells with MTT solution at 0.5 mg/mL and conducting reaction at 37° C. for 2 hours, the resulting formazan was dissolved with DMSO and absorbance was measured at a wavelength of 570 nm using a microplate reader. Cell viability was calculated according to Equation 3 as a ratio with respect to the control group.

$$Cell viability = absorbance of treatment group / absorbance of control group \qquad [ Equation 3 ]$$

Human keratinocytes (HaCaT) were seeded into a 24-well plate at 5×104 cells/well. 24 hours later, the cells were treated with the sample at different concentrations after replacing the medium with a serum-free DMEM medium. After 24 hours of incubation, the cell culture was removed and centrifuged at 15,000 g for 5 minutes. The supernatant was removed to quantify hyaluronic acid using an R&D Quantikine hyaluronan ELISA kit. The change in hyaluronic acid synthesis was calculated according to Equation 4 as a ratio with respect to the control group

$$Hyaluronic acid = absorbance of treatment group / absorbance of control group \qquad [ Equation 4 ]$$

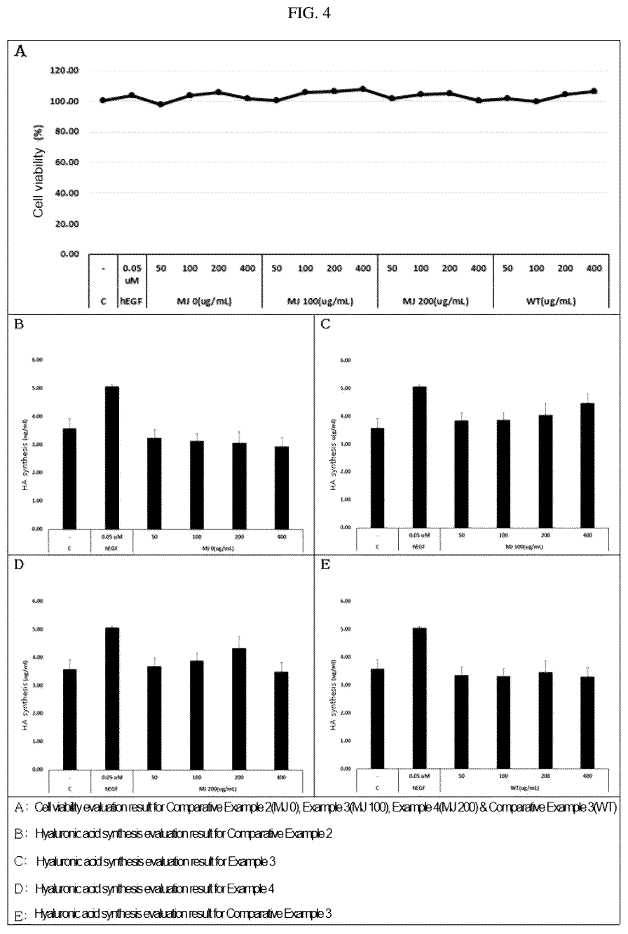

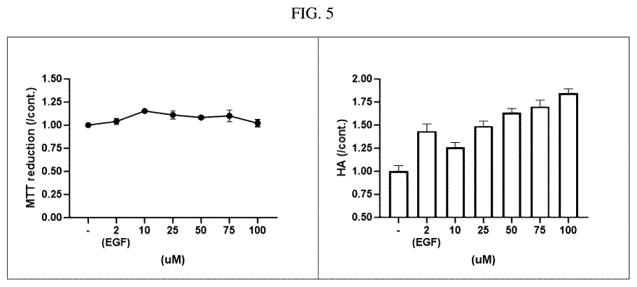

FIG. 4 shows a result of investigating cell viability and hyaluronic acid synthesis in Test Example 5. A result of quantifying the result shown in FIG. 4 is given in Table 4. FIG. 5 shows a result of evaluating the synthesis of hyaluronic acid depending on the concentration of digiferruginol-11-O-β-primeveroside isolated in Test Example 3.

TABLE 4

| | Treatment | Cell | Hyaluronic |
|---|---|---|---|
| | concentration | viability | acid synthesis |
| Sample | (μg/mL) | (%) | (μg/mL) |
| Untreated group | 0 | 100 ± 0.02 | 3.58 ± 0.35 |
| Comparative Example 2 | 50 | 97.3 ± 0.03 | 3.23 ± 0.35 |
| Comparative Example 2 | 100 | 103.2 ± 0.01 | 3.12 ± 0.11 |
| Comparative Example 2 | 200 | 104.9 ±0.03 | 3.04 ± 0.30 |
| Comparative Example 2 | 400 | 100.9 ± 0.01 | 2.93 ± 0.10 |
| Example 3 | 50 | 100.0 ± 0.02 | 3.84 ± 0.43 |
| Example 3 | 100 | 105.6 ± 0.00 | 3.85 ± 0.17 |
| Example 3 | 200 | 105.8 ± 0.01 | 4.04 ± 0.60 |
| Example 3 | 400 | 107.5 ± 0.02 | 4.47 ± 0.60 |
| Example 4 | 50 | 101.1 ± 0.02 | 3.68 ± 0.29 |
| Example 4 | 100 | 103.9 ± 0.01 | 3.88 ± 0.34 |
| Example 4 | 200 | 104.2 ± 0.01 | 4.33 ± 0.54 |
| Example 4 | 400 | 99.6 ± 0.05 | 3.48 ± 0.51 |
| Comparative Example 3 | 50 | 101.3 ± 0.04 | 5.05 ± 0.30 |
| Comparative Example 3 | 100 | 99.5 ± 0.04 | 3.34 ± 0.28 |
| Comparative Example 3 | 200 | 104.1 ± 0.02 | 3.32 ± 0.43 |
| Comparative Example 3 | 400 | 105.8 ± 0.01 | 3.46 ± 0.34 |

### [Analysis of Results]

As seen from the above experimental results, the content of digiferruginol-11-O-β-primeveroside was increased by 46 times and the synthesis of hyaluronic acid was also increased by 1.2 times in the Damnacanthus major callus treated with methyl jasmonate at 100 UM, as compared to the group not treated with methyl jasmonate. Damnacanthus major has a very small population in Korea, and its biomass is very scarce for industrial application. It is expected that the method for culturing the callus of Damnacanthus major developed in the present disclosure, which provides increased digiferruginol-11-O-β-primeveroside content and increased moisturizing effect, is industrially applicable through mass production.

With reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A method of cultivating and extracting callus of Damnacanthus major comprising:
a first culturing step of inoculating the callus of Damnacanthus major to a culture medium comprising a MS (Murashige & Skog) salt, a mixture of α-naphthaleneacetic acid and kinetin as a plant growth regulator, and sucrose, and culturing Damnacanthus major callus cells at a temperature of 24 to 26° C.;
a second culturing step of sequentially treating the callus of Damnacanthus major with methyl jasmonate as an elicitor so that the concentration of the methyl jasmonate in the culture medium is maintained at 70 to 150 UM; and
a step of extracting the cultured Damnacanthus major callus with hot water and separating a Damnacanthus major callus extract; and a step of decompression-concentrating and freeze-drying the separated callus extract,
wherein the Damnacanthus major callus extract comprises at least 1.8 mg/g of digiferruginol-11-O-β-primeveroside as an active ingredient.

2. The method of claim 1, wherein the pH of the culture medium is adjusted to 5.7 to 5.8.
